(19) United States
(12) Patent Application Publication
(10) Pub. No.: US 2013/0071396 A1
(43) Pub. Date: Mar. 21, 2013

(54) METHOD OF INHIBITING COAGULATION ACTIVATION WITH HUMAN ANTI-FACTOR V ANTIBODIES AND USE THEREOF

(71) Applicant: Rekha Bansal, Twinsburg, OH (US)

(72) Inventor: Rekha Bansal, Twinsburg, OH (US)

(21) Appl. No.: 13/428,611

(22) Filed: Mar. 23, 2012

Related U.S. Application Data
(63) Continuation of application No. 12/445,864, filed on Sep. 8, 2010, now abandoned.
(60) Provisional application No. 60/852,032, filed on Oct. 16, 2006.

(51) Int. Cl. A61K 39/395

(52) U.S. Cl. CPC A61K 39/3955
USPC 424/135.1; 424/158.1; 424/133.1

(57) ABSTRACT

The present invention also discloses the novel use of factor Va inhibitors in the treatment of various disorders caused by the formation of blood clots.

### FIELD OF INVENTION

The present invention relates to inhibition of pathological thrombin formation as a result of activation of the coagulation cascade by monoclonal antibody to factor Va. Particularly, the present invention relates to a method for blocking thrombin production by inhibiting prothrombinase complex formation.

### BACKGROUND

Under conditions of clinical insult, blood thickens and gradually becomes a clot. This process of clot formation is generally considered a part of the normal physiological process and is important to stop unnecessary bleeding during blood vessel damage. Blood coagulation occurs through a complex series of molecular reactions, ultimately resulting in the conversion of soluble fibrinogen molecules into insoluble threads of fibrin. This process results in a blood clot, which consists of a plug of platelets entangled in the fibrin network. The coagulation system functions to prevent the loss of blood after injury. Interactions between activated platelets and coagulation proteins are critical for the maintenance of normal hemostasis.

The coagulation cascade is initiated by at least two different pathways; a) the process of contact activation (intrinsic pathway), and b) the action of tissue factor (extrinsic pathway). Activation of either initiating pathway leads to activation of the common coagulation pathway. The common pathway of coagulation begins with the activation of factor X to Xa. The interaction of factor Xa with factor Va, Ca²⁺, and phospholipids results in activation of prothrombin to thrombin. The complex phospholipid-Va-Xa is called prothrombinase. Both, intrinsic and extrinsic pathways converge at the central point of factor X activation. Regardless of the pathway of activation, factor Xa is produced as a result of activation of either the intrinsic or the extrinsic pathways to initiate the coagulation cascade. Activated factor Va binds factor Xa with high affinity to generate prothrombinase. Prothrombinase cleaves prothrombin (factor II) to yield thrombin (IIa). Thrombin's role in the coagulation cascade is several folds. Thrombin is known to activate platelets and clotting factors. Thrombin converts fibrinogen into fibrin and leads to clot formation. Fibrinogen is a dimer that is soluble in plasma. Exposure of fibrinogen to thrombin results in rapid proteolysis of fibrinogen and the release of fibrinopeptide A (FPA). The loss of small peptide A is not sufficient to render the resulting fibrin molecule insoluble, a process that is required for clot formation, but it tends to form complexes with adjacent fibrin and fibrinogen molecules. A second peptide, fibrinopeptide B, is then cleaved by thrombin, and the fibrin monomers formed by this second proteolytic cleavage polymerize spontaneously to form an insoluble gel. In vivo, FPA is used as a marker to determine the rate of conversion of fibrinogen to fibrin by thrombin. An increased FPA level (>3 ng/ml), indicates the existence of an excess of thrombin activity. FPA is elevated in many clinical situations associated with blood activation, evolutive thrombosis, and malignancies. It is therefore a marker of hypercoagulable states induced in these pathological conditions.

Prothrombinase is required for the normal clotting function. It is composed of factors Xa and Va which associate on phospholipids (on platelet surface) in the presence of divalent metal ions. Factor Va, the non-enzymatic subunit, does not by itself cleave thrombin, but increases the cleavage activity of factor Xa by 300,000 times. In the blood, thrombin cleaves factor V to produce the factor Va. Unlike thrombin, which acts on a variety of protein substrates as well as at a specific receptor, factor Xa appears to have a single physiologic substrate, prothrombin. Studies have shown that factor Va and factor Xa binding can occur both in the presence and absence of phospholipids (1-3) but the activity of Xa-Va complex increases several folds in the presence of phospholipids (4). Factor Va (5) is derived from the pro-cofactor, factor V, upon limited proteolysis by thrombin (6). Factor Va is comprised of an NH₂-terminal derived heavy chain (Mr=94,000) and a COOH-terminal derived light chain (Mr=74,000) which remain associated in the presence of calcium ions. Factor Va is a cofactor for the serine protease factor Xa, and in the presence of calcium ions it collectively assembles on a phospholipid surface to form the prothrombinase complex Factor Va (6), composed of a heavy (Va_{H}) and light (Va_{L}) chain and binds to factor Xa (7) in a stoichiometric manner. The interaction between factor Va (8) and factor Xa is mediated by both the heavy and light chain of factor Va, while the binding of prothrombin to factor Va is mediated solely by the heavy chain. Factors Xa and Va interact stoichiometrically in the presence of phospholipids.

Prothrombinase complex represents the point at which the intrinsic and extrinsic blood coagulation pathways converge and is an ideal point for an anticoagulant molecule to act since inhibition at this point blocks both intrinsic and extrinsic pathways (FIG. 1). Prothrombinase cleaves prothrombin to form thrombin, a central step in blood coagulation. Thrombin is a well-known agonist of platelets and leads to platelet activation. After activation, platelets accelerate the generation of thrombin by providing an effective phospholipid catalytic surface for the conversion of prothrombin to thrombin as shown in the cascade. This conversion is mediated by factors Xa and Va which bind platelet surface with high affinity. Thus it appears that anionic phospholipids (on the platelet surface) (9) are required for formation of this binding site and that inhibition of the assembly of prothrombinase is key for efficiently blocking blood coagulation and further platelet activation via thrombin.

The overall balance between coagulants and anticoagulants determine whether blood will clot. Under normal hemostasis, balance is always in favor of the anticoagulants. However, in response to injury or trauma, this balance shifts to favor coagulants and blood clots are invariably formed. Plasmin reacts very quickly to dissociate the clot. Circulating blood contains plasminogen which binds fibrin molecules within the blood clot. Tissue Plasminogen Activator (TPA) which binds to fibrin, which is subsequently activated and cleaves plasminogen to plasmin Plasmin cleaves fibrin and the clot is dissolved.

Under abnormal conditions, however, blood clots are observed within the blood. Such blood clots are formed as a result of a clinical disorder called “thromboses”. There are two types of thromboses. The first, arterial thrombosis, is caused by occlusion of arteries, which leads to myocardial infarction, unstable angina, arterial fibrillation, stroke, renal damage, percutaneous transluminal coronary angioplasty, intravascular coagulation, sepsis, artificial organs, shunts and prosthesis and peripheral ischemia. The second type is venous thrombosis. Venous thrombosis is caused by the occlusion of venous blood vessels and results in pulmonary emboli (PE) and deep vein thrombosis (DVT). In order to prevent or treat such thrombotic disorders, therapeutic methods to inhibit clot formation or to dissolve clots have been developed. Existing anticoagulants, warfarin and heparin have been in clinical use for about 50 years. Nonetheless, both are associated with several well-documented drawbacks that limit their usefulness. Widely used heparin has a variable dose response relationship due to its non-specific binding to plasma proteins, platelets, hepatic macrophages and bone cells that necessitate frequent coagulation monitoring. Additionally, heparin treatment can result in osteoporosis and thrombocytopenia. These drawbacks have created a need for new and improved antithrombotic agents. Several new anticoagulants are currently being developed or are in clinical trials for inhibiting coagulation.

For example, TFPI and NAPc2 prevent initiation of coagulation by acting on factor VIIa/tissue factor complexes. APC prevents generation of factor VIIIa and factor Va. None have been approved by the FDA due to safety/toxicity issues. There appears to be a series of factor Xa inhibitors discovered in the last ten years but only a few have recently made it to phase II trials. Both, antistatin (ATS, isolated from the Mexican leech), and tick anticoagulant peptide (TAP, isolated from ornithidoros moubata) are potent inhibitors of factor Xa activity but due to immunogenicity issues, could not be developed into therapeutics. The anticoagulant pentasaccharides DX-9065a and DPC-906 directly inhibit factor Xa activity (10), however, there is a lack of simple tests to monitor their efficacy, which has limited their potential use as successful inhibitors clinically. Additionally some synthetic inhibitors of factor Xa activity have also been discovered but none have gained FDA approval due to safety and toxicity issues. The only drug that has gained FDA approval is Angiomax (Bivaluridin), which a peptide and long-term effects of this peptide drug are not known.

Thrombin acts as a catalyst for converting fibrinogen to fibrin, which subsequently cross-links to form the mesh that creates a thrombus. Direct or indirect inhibition of thrombin activity (11) has been the focus of a variety of recent anticoagulant strategies. Several classes of the currently used anticoagulants either directly or indirectly inhibit thrombin activity (i.e. heparins, low-molecular weight heparins, heparin-like compounds and coumarins). These inhibitors have low potency and thus high concentration of the drug is needed to inhibit the coagulation cascade. Given the unique role of thrombin in the coagulation cascade, its inhibition is key to successful antithrombotic pharmacotherapy (12, 13). Antithrombotic drugs are classified as direct thrombin inhibitors (DTIs); indirect thrombin inhibitors (eg, UFH, low molecular weight heparin [LMWH], fondaparinux); thrombin-generation inhibitors (eg, f Xa inhibitor, inactivated f X); or recombinant endogenous anticoagulants (eg, activated protein C, antithrombin, heparin cofactor II). Other anti-coagulants such as Hirudin, Bivalirudin, Argatroban, and Ximelgatran (14) are direct inhibitors of thrombin. These agents bind one or both catalytic sites present on thrombin making it inactive. Thrombin inhibitors have the following limitation; one mole of prothrombinase generates several moles of thrombin and thus a large excess of thrombin inhibitors are required to inhibit thrombin action. Thus, the prothrombin to thrombin step is an amplification point in pathway. Inhibitors that prevent thrombin production are therefore desirable because they target the pathway prior to the amplification step and as such should require a much lower concentration of the inhibitor for potent inhibition of coagulation. Antibodies to factor Va have been developed (15, 16). Interactions of Va and ctr-EGR-Xa have also been investigated using monoclonal antibodies to factor Va.

Inhibition of thrombin requires a large quantity of the inhibitory drugs compared to drugs that inhibit prothrombinase, a step that occurs prior to the amplification point in the pathway for thrombin production. An ideal drug that prevents blood clot formation would target a single clotting factor such that side effects resulting from nonspecific action of the drug are minimized or eliminated. Such ideal drugs would have superior efficacy and safety profiles since thromboses would be inhibited without bleeding as a side effect. Additionally, because of the many different manifestations and etiologies of thrombosis, and the different locations in the body where clots can form, there is a need for new and varied treatments for these manifestations. The development of a monoclonal antibody to factor Va would be of added advantage because antibodies are highly target specific and are used at low concentration. Thus, a monoclonal antibody against factor V/Va should efficiently block the coagulation pathway at a step just prior to the generation of thrombin at low concentrations and with good safety profiles due to its target specificity.

It is to be noted that throughout this application various publications are referenced by Arabic numerals within brackets. Full citations for these publications are listed at the end of the specification. The disclosures of these publications are hereby incorporated by reference in their entireties into this application in order to more fully describe the state of the art to which this invention pertains.

### SUMMARY OF INVENTION

In accordance with the present invention, it has been discovered that monoclonal antibodies against factor V/Va exhibit complete inhibition of thrombin production. The antibody is effective at low nM concentration and inhibits thrombin and FPA/FPB production. The present invention also provides new processes for reducing and preventing unwanted clotting of blood arising from clinical situations and clinical procedures in mammals, including humans, comprising administering these pharmaceutical compositions to the mammals.

The present invention also provides a method of inhibiting FPA and FPB production from activation of coagulation pathways. The process includes the step of inhibiting thrombin production, which ultimately prevents fibrin formation that results in clot formation. Thrombin-induced formation of fibrin is inhibited by factor Va antibody inhibition of the binding of factor Va to factor Xa. The binding of factor Va to factor Xa is inhibited by exposing factor Va to an effective amount of an antibody against factor Va. Accordingly, the invention discloses a novel method of inhibiting coagulation activation by inhibiting the activity of factor V that is responsible for the formation of Xa-Va or phospholipid-Xa-Va complexes.

Factor V inhibitor molecules comprise whole or fragmented anti-factor V antibodies having a binding region specific to factor Va. Antibody fragments can be F_{ab}, F_{(ab)2}, Fᵥ, or single chain Fᵥ. The antibody may be monoclonal, polyclonal, chimeric, recombinant or De-Immunized. The inhibitor molecule of the present invention can prevent factor V binding to platelet factor 3 (PF3) bound Xa, inhibit the assembly of prothrombinase; or prevent the cleavage of factor V into Va.

The present invention also discloses the use of factor V inhibitors for the treatment of several disease conditions involving coagulation activation. These include the treatment and prevention of arterial and venous thromboses. Clinical indications for arterial thromboses include to myocardial infarction (MI), acute coronary syndromes (ACS), stroke, and peripheral embolization. Clinical indications for venous thrombosis may manifest as acute deep vein thrombosis (DVT), pulmonary embolism (PE), and paradoxical arterial embolization. Also included in clinical indications are surgical procedures that may complicate the performance of cardiovascular procedures or initiate malfunction of foreign devices implanted in the cardiovascular system (heart valves, arterial stents, venous filters, bypass grafts, etc). Other clinical situations covered by such invention are post cardiopulmonary bypass complications, deep vein thrombosis, ischemia/reperfusion injury stroke, acute respiratory distress syndrome (ARDS), inflammation associated with cardiopulmonary bypass and hemodialysis, plasmapheresis, plateletpheresis, leukophereses, extracorporeal, membrane oxygenation (ECMO), heparin-induced extracorporeal LDL precipitation (HELP). In vivo inhibition of coagulation activation is accomplished by administering the anti-Va antibody to the subject. Inhibition of coagulation is also accomplished by administering anti-V/Va antibodies to blood in extra-corporeal circulation. Pharmaceutical compositions containing anti-Va antibodies are also provided.

Anticoagulant therapy is indicated for the treatment and prevention of a variety of thrombotic conditions, particularly coronary artery and cerebrovascular disease. Those experienced in this field are readily aware of the circumstances requiring anticoagulant therapy.

Thrombin inhibition is useful not only in the anticoagulant therapy of individuals having thrombotic conditions, but is useful whenever inhibition of blood coagulation is required, such as to prevent coagulation of stored whole blood and to prevent coagulation in other biological samples for testing or storage. Thus, the thrombin inhibitors can be added to or contacted with any medium containing or suspected of containing thrombin and in which it is desired that blood coagulation be inhibited, e.g., when contacting the mammal's blood with material selected from the group consisting of vascular grafts, stents, orthopedic prosthesis, cardiac prosthesis, and extracorporeal circulation systems.

Antibodies of the invention are useful for treating or preventing venous thromboembolism (e.g. obstruction or occlusion of a vein by a detached thrombus; obstruction or occlusion of a lung artery by a detached thrombus), cardiogenic thromboembolism (e.g. obstruction or occlusion of the heart by a detached thrombus), arterial thrombosis (e.g. formation of a thrombus within an artery that may cause infarction of tissue supplied by the artery), atherosclerosis (e.g. arteriosclerosis characterized by irregularly distributed lipid deposits) in mammals, and for lowering the propensity of devices that come into contact with blood to clot blood.

Examples of venous thromboembolism which may be treated or prevented with monoclonal antibodies of the invention include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, compounds of the invention are useful for maintaining patency of indwelling catheters.

Examples of cardiogenic thromboembolism which may be treated or prevented with antibodies of the invention include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

Examples of arterial thrombosis include unstable angina (severe constrictive pain in chest of coronary origin), myocardial infarction (heart muscle cell death resulting from insufficient blood supply), ischemic heart disease (local anemia due to obstruction (such as by arterial narrowing) of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterial thrombosis, compounds of the invention are useful for maintaining patency in arteriovenous cannulas.

Examples of devices that come into contact with blood include vascular grafts, stents, orthopedic prosthesis, cardiac prosthesis, and extracorporeal circulation systems.

The present invention provides, in one aspect, a process of inhibiting the adverse effects of coagulation pathway activation in a subject by administering to the subject an amount of an anti-V or anti-Va agent effective to selectively inhibit formation (i.e., generation or production) of a coagulation activation product. Formation of such intrinsic pathway-dependent coagulation activation products refers to the generation or production of such products by coagulation activation, which products when generated or produced can be detected. These products include the intrinsic pathway-dependent thrombin and fibrin products produced with activation of the coagulation pathway. An anti-factor V agent according to the invention blocks factor Va binding to Xa as described herein and inhibits the formation of thrombin and fibrin. Such agents include an anti-factor Va antibody, an antigen-binding fragment of an anti-factor Va antibody, and a factor V derived peptide. Preferably, the anti-factor Va agent does not substantially activate Fc gamma receptors and/or the complement pathway.

The present invention provides, in another aspect, a process for inhibiting the adverse effects of extrinsic coagulation pathway activation in a subject in which the extrinsic coagulation pathway is initiated by cellular damage or by tissue factor. The anti-factor Va agent is effective to selectively inhibit formation of thrombin and fibrin under conditions of cellular damage or by intrinsic or extrinsic pathway.

The present invention provides, in another aspect, an article of manufacture comprising packaging material and a pharmaceutical agent (i.e., pharmaceutical composition) contained within the packaging material, wherein: (a) the pharmaceutical agent comprises an anti-factor Va agent, the anti-factor Va agent being effective for reducing at least one of coagulation activation, platelet activation, leukocyte activation, or platelet adhesion caused by passage of circulating blood from a blood vessel of a subject, through a conduit, and back to a blood vessel of the subject, the conduit having a luminal surface comprising a material capable of causing at least one of complement activation, platelet activation, leukocyte activation, or platelet-leukocyte adhesion in the subject's blood; and (b) the packaging material comprises a label which indicates that the pharmaceutical agent is for use in association with an extracorporeal circulation procedure.

The invention provides for the use of an anti-factor Va agent in the preparation of a medicament for selectively inhibiting formation of coagulation activation products via the intrinsic coagulation pathway in a subject in need thereof. Also provided is for the use of an anti-factor Va agent in the preparation of a medicament for selectively inhibiting formation of coagulation activation products via the intrinsic coagulation pathway in a subject in which the extrinsic pathway is initiated. Additionally provided is for the use of an intrinsic coagulation pathway prothrombinase inhibiting agent in the preparation of a medicament for inhibiting formation of coagulation activation products.

### BRIEF DESCRIPTION OF DRAWINGS

FIG. 1 illustrates a schematic drawing of both, Intrinsic and extrinsic pathways. The diagram shows that inhibition of factor Va association with factor Xa is important for theombin formation via both pathways. If this association is blocked, formation of thrombin can be prevented. We have selected eight monoclonal antibodies against human and bovine factor V or Va and tested them for thrombin production via the intrinsic/extrinsic pathways.

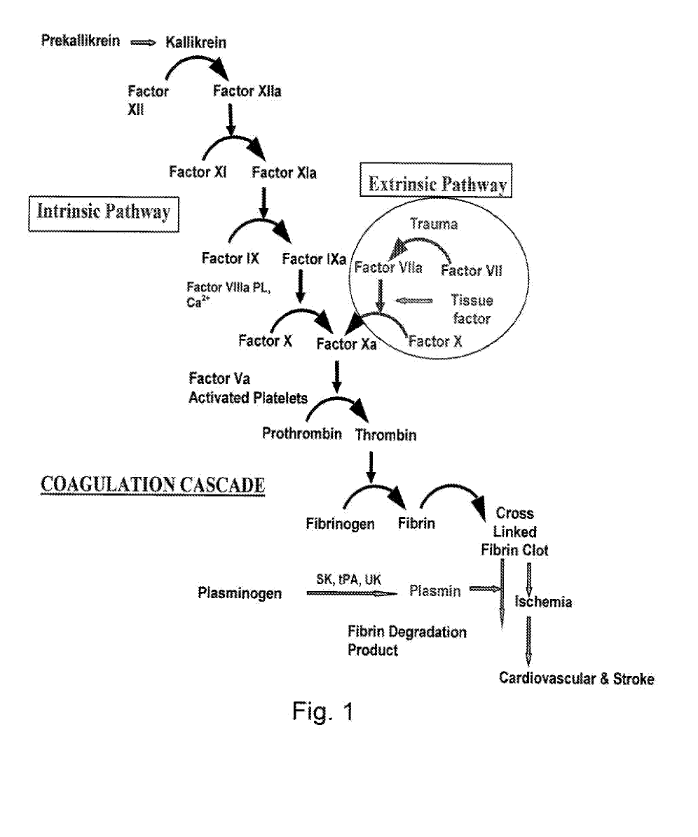

FIG. 2: Binding assay demonstrating that human anti-factor V/Va monoclonal antibodies bind to substrate-bound factor Va at 1:2000 dilution. ELISA plates were coated with 20 ng/50 ul Factor Va per well and incubated overnight in cold. The plates were blocked with 1% BSA in PBS for 1 hour. Anti-Factor V/Va monoclonal antibodies in blocking solution at 1:2000 dilutions were incubated with substrate bound factor Va. Following a 1 hour incubation at room temperature, the plate was rinsed and the bound anti-Factor V/Va monoclonal antibodies were detected with peroxidase-conjugated goat anti-mouse antibody (Sigma Chemical Company) at 1:2000 dilution. The plate was washed and incubated with 100 ul of TMB substrate for 10 minutes. The plate was read at 450 nm after quenching with 100 ul aliquots of 1 M phosphoric acid.

FIG. 3: Assay of levels of thrombin generation in citrated human plasma via the Tissue factor Pathway demonstrating that the generation of thrombin can be inhibited by the addition of an anti-factor V monoclonal antibody that binds factor Va. In the assay, all eight anti-factor V antibodies in 10% normal human plasma were incubated with chromogenic substrate S2238 at 37° C. Innovin (a PT reagent from Dade Behring) was added and the production of thrombin was measured as a function of time. As shown in the figure, the Y-axis represents the level of thrombin generated and the X-axis represents the time of incubation. FIG. 3 shows thrombin production as a function of time. In this assay, an OD of 1.5 represents maximum thrombin production. Normal Human Plasma containing chromogen S-2238 was activated with Innovin (activator with Ca++) in the presence of various anti-Va antibodies. Heparin (Open square, the first line) was used as a positive control. Only one antibody blocked the formation of Thrombin (second curve from the bottom, open triangle). This selected antibody was characterized as a blocking antibody

FIG. 4: Assay of levels of thrombin generation in citrated human plasma, via the extrinsic pathway (PT, TF), demonstrating that the anti-factor V mediated inhibition of thrombin is dose dependent. In the assay, anti-factor V antibody at various concentrations (10 ug/ml, 5 ug/ml, 2.5 ug/ml, and 1.25 ug/ml) in 10% normal human plasma was incubated with chromogenic substrate S2238 at 37° C. Innovin (PT reagent from Dade Behring) was added and the production of thrombin was measured as a function of time. As shown in the figure, the Y-axis represents the level of thrombin generated and the X-axis represents the time of incubation. At a concentration of 10 ug/ml the anti-factor Va antibody caused a complete inhibition of thrombin production in 10% normal citrated human plasma. FIG. 4 shows the dose dependent inhibition of Thrombin production by various concentrations of the selected anti-V monoclonal antibody. In a typical setting, normal human plasma containing S-2238 was mixed with various doses of the selected anti-factor V antibody. The plasma mix was treated with an extrinsic pathway activator Innovin (Dade Behring). The progression of thrombin formation was measured as a function of time. The open triangle is heparin control showing inhibition of thrombin formation, the second line from the bottom is 100 ug/ml of anti-Va, the third line is 50 ug/ml, the fourth line is 25 ug/ml, and the fifth line represents untreated control demonstrating maximum thrombin production in the assay. The selected anti-V inhibits thrombin formation.

FIG. 5: This Figure shows the dose dependent inhibition of thrombin production (via the intrinsic pathway Contact activation, aPTT, FSL) by various concentrations of the selected anti-V monoclonal antibody (from FIG. 3) in the intrinsic pathway activation. Normal human plasma containing TGA (technothrombin, Technolcone, Inc.) was mixed with various doses of anti-V8 antibodies. The human plasma (20%) with and without anti-factor V monoclonal antibody containing was activated with Actin FSL (aPTT reagent, activator from Dade Behring) and the amount of thrombin production was monitored with TGA (Fluorescent substrate for thrombin). The thrombin production was measured with time in a Gemini XS fluorescence temperature controlled ELISA plate reader. TGA (Z-GGR-AMC) is a known substrate for thrombin and when cleaved generates fluorescence which is measured over time. The open square at the bottom line is heparin control, the second line from the bottom is 200 ug/ml of anti-Va, the third line is 150 ug/ml, the fourth line is 75 ug/ml, the fifth line is 37.5 ug/ml, the sixth line is 18.75 ug/ml, and the seventh line is 9.38 ug/ml, the eighth line is 4.5 ug/ml, and the ninth line is 2.25 ug/ml. The 10^{th} line served as a negative control. The monoclonal antibody anti-factor V (FIG. 3) inhibits contact activation by 50% at a concentration of 200 ug/ml in citrated 20% normal human plasma. The unfractionated heparin was used as a positive control, which totally inhibited thrombin production.

FIG. 6: Binding assay demonstrating that human factor Va binds to human factor Xa with high affinity. The vertical Y-axis represents the reactivity of the factor Va with Xa and horizontal X-axis represents the concentration of factor Va. In this assay, the ELISA plate was coated with 20 ng/50 ul of factor Xa (Haematologic Technologies). The plate was blocked with 1% BSA solution. After washing with PBS, the plate was incubated with varying concentrations of Factor Va. The Va was detected with non blocking anti-factor Va antibody as described above in FIG. 2. In the bottom panel is shown the dose-dependent inhibition of factor Va binding to substrate bound Xa using the selected blocking anti factor V monoclonal antibody. The Y-axis represents the inhibition of factor Va binding, and the X-axis represents the concentration of anti-factor V antibody. The assay is similar to the binding assay. In this assay, a constant concentration of Factor Va was incubated with 100 ug/ml concentration of anti-Factor V antibody. The bound Factor Va was detected with anti-factor VVa antibody as described above.

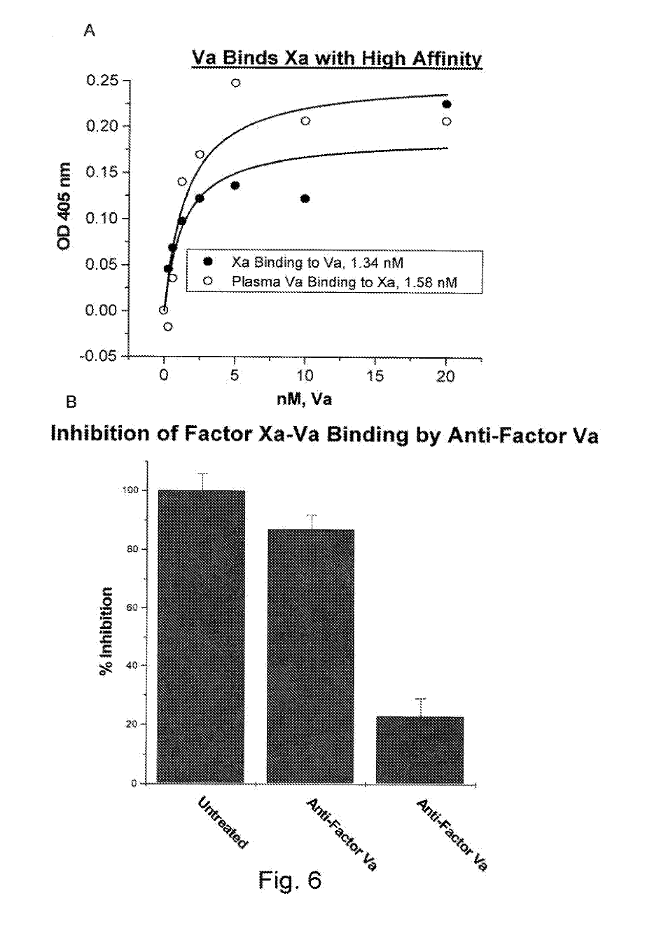

As shown in FIG. 6 top panel, closed circle shows the binding interactions of Xa and Va in the presence of phospholipids. Open circles show binding interactions of Xa (pure protein added) and Va (endogenous) in factor X depleted plasma where factor V was activated with RVV-V (Russel Viper Venom) to convert V into Va for efficient binding to factor Xa. Notice that ELISA wells were coated with factor Xa prior to incubation with Va alone or a plasma containing freshly activated Va. Factor Va in plasma (or pure protein Va) binds substrate-bound factor Xa with nM affinity. In the bar graph are shown the effects of anti-factor V monoclonal antibody addition to factor Va in the presence of phosphorlipid vesicles, and 1 mM calcium. The mixture was incubated with substrate-bound factor Xa. Two different concentrations of factor V antibodies were used: the first column is total binding, the second column is 100 ug/ml of anti-factor V monoclonal antibody and the third column contains 200 ug/ml of the monoclonal antibody. Anti-Factor V monoclonal antibody inhibits Factor Va binding to Substrate-bound Xa

### DETAILED DESCRIPTION

The present invention discloses the new use of anti-factor V/Va monoclonal antibody for inhibiting thrombin formation via extrinsic and intrinsic pathways of coagulation in various disease conditions that involves: (a) inhibiting cleavage of Factor V into Va, (b) inhibiting factor Va binding to phospholipid-bound Xa on platelets; (c) inhibiting the conversion of prothrombin into thrombin, (d) inhibiting the release of fibrinopeptide A; (e) inhibiting the activation of leukocytes and platelets; (f) inhibiting/reducing the formation of complex phospholipid-Xa-Va, thrombin and fibrin in clinical conditions where the disease pathology is mediated via thrombin production and fibrin formation. The present invention also discloses the novel use of factor V/Va monoclonal antibody inhibitors for the treatment of many acute disorders where blood clot formation is considered pathological. The diseases treated by factor V/Va inhibitors include, but are not limited to myocardial infarction, ischemia/reperfusion injury, vascular stenosis or post-angioplasty restenosis, stroke, acute respiratory distress syndrome (ARDS), deep vein thrombosis, cardiopulmonary bypass inflammation, and extracorporeal circulation such as hemodialysis, plasmapheresis, platelet pheresis, leukopheresis, extracorporeal membrane oxygenation (ECMO), or heparin-induced extracorporeal LDL precipitation (HELP).

Anti-factor V/Va monoclonal antibodies can be prepared by standard methods well known in the art. For example, rodents (e.g. mice, rats, hamsters, and guinea pigs) can be immunized either with native factor V or Va purified from human plasma or with recombinant factor V or its fragments expressed by either eukaryotic or prokaryotic systems. Other animals can also be used for immunization, e.g. non-human primates, transgenic mice expressing human immuno-globulins, and severe combined immuno-deficient mice transplanted with human V-lymphocytes. Hybridoma can be generated by conventional procedures well known in the art by fusing B lymphocytes from the immunized animals with myeloma cells (e.g. Sp2/0 and NS0). In addition, anti-factor V/Va antibodies can be generated by screening of recombinant single-chain Fᵥ or F_{ab} libraries from human B lymphocytes in phage-display systems. The specificity of the monoclonal antibodies to human factor V can be tested by enzyme linked immuno-sorbent assay (ELISA).

It would be evident to ones skilled in the art that in vitro studies of coagulation are representative of and predictive of the in vivo state of the coagulation system. By way of example, the use of an in vitro chromogenic procedure to detect thrombin is a simple, rapid and reliable method for the assessment of coagulation function. Thus, the in vitro technique can be used in vivo with the same likelihood of success in detecting coagulation activation in disease states. Furthermore, the standard thrombin based chromogenic assay is accepted in the art as being the “most convenient” assay for determining the activity of the human coagulation pathway.

To prevent platelet activation that occurs due to other factors known in the art, anti-platelet agents covering GP11bIIIa antagonists and aspirin like molecules can be administered in combination with the anti-V or anti-Va antibody. In some cases, combination therapy lowers the therapeutically effective dose of anti-coagulation factor Va monoclonal antibody.

Thus, the molecules of the present invention, when in preparations and formulations appropriate for therapeutic use, are highly desirable for abnormal clotting activity associated with, but not limited to, myocardial infarction, unstable angina, atrial fibrillation, stroke, renal damage, pulmonary embolism, deep vein thrombosis and artificial organ and prosthetic implants.

The present invention provides a variety of antibodies, including chimeric, engineered, humanized, fully human, and altered antibodies and fragments thereof directed against factors V and Va. The antibodies of the present invention can be prepared by conventional hybridoma techniques, phage display combinatorial libraries, immunoglobulin chain shuffling and humanization techniques to generate novel neutralizing antibodies. Also included are fully human monoclonal antibodies with inhibitory activity. These products are useful in therapeutic and pharmaceutical compositions for thrombotic and embolic disorders associated with several clinical indications outlined in claims section. As used herein, the term “inhibitory activity” refers to the activity of an antibody that inhibits thrombin production and FPA formation in whole blood.

“Altered antibody” refers to chimeric or humanized antibodies or antibody fragments lacking all or part of an immunoglobulin constant region, e.g., Fv, Fab, Fab′ or F(ab′)₂ and the like.

As used herein, an “engineered antibody” describes a type of altered antibody, i.e., a full-length synthetic antibody (e.g., a chimeric or humanized antibody as opposed to an antibody fragment) in which a portion of the light and/or heavy chain variable domains of a selected acceptor antibody are replaced by analogous parts from one or more donor antibodies which have specificity for the selected epitope.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs (spell out abbreviation at first use) derived from a non-human donor immunoglobulin, the remaining immunoglobulin-derived parts of the molecule being derived from one or more human immunoglobulins. In addition, framework support residues may be altered to preserve binding affinity.

A “functional fragment” is a partial heavy or light chain variable sequence (e.g., minor deletions at the amino or carboxy terminus of the immunoglobulin variable region) which retains the same antigen binding specificity and/or neutralizing ability as the antibody from which the fragment was derived.

An “analog” is an amino acid sequence modified by at least one amino acid, wherein said modification can be chemical or a substitution or a rearrangement of a few amino acids (i.e., no more than 10), which modification permits the amino acid sequence to retain the biological characteristics, e.g., antigen specificity and high affinity, of the unmodified sequence. Exemplary analogs include silent mutations which can be constructed, via substitutions, to create certain endo-nuclease restriction sites within or surrounding CDR-encoding regions.

Analogs may also arise as allelic variations. An “allelic variation or modification” is an alteration in the nucleic acid sequence encoding the amino acid or peptide sequences of the invention. Such variations or modifications may be due to degeneracy in the genetic code or may be deliberately engineered to provide desired characteristics. These variations or modifications may or may not result in alterations in any encoded amino acid sequence.

The term “effector agents” refers to non-protein carrier molecules to which the altered antibodies, and/or natural or synthetic light or heavy chains of the donor antibody or other fragments of the donor antibody may be associated by conventional means. Such non-protein carriers can include conventional carriers used in the diagnostic field, e.g., polystyrene or other plastic beads, polysaccharides, e.g., as used in the BIAcore (Pharmacia) system, or other non-protein substances useful in the medical field and safe for administration to humans and animals. Other effector agents may include a macrocycle, for chelating a heavy metal atom or radioisotopes. Such effector agents may also be useful to increase the half-life of the altered antibodies, e.g., polyethylene glycol.

For use in constructing the antibodies, altered antibodies and fragments of this invention, a non-human species such as bovine, ovine, monkey, chicken, rodent (e.g., murine and rat) may be employed to generate a desirable immunoglobulin upon presentment with a human factor V and factor Va. Conventional hybridoma techniques are employed to provide a hybridoma cell line secreting a non-human monoclonal antibody to the respective coagulation factor. Such hybridomas are then screened for thrombin generation and FPA using Innovin—an activator of the extrinsic pathway. Alternatively, fully human monoclonal antibodies can be generated by techniques known to those skilled in the art and used in this invention.

In an aspect of the invention, the anti-factor V/Va antibody can be specific to the heavy chain of the factor Va peptide. In another aspect, the anti-factor V/Va antibody can be specific to peptides 307 to 348 of the factor Va chain and/or peptides 323 to 331 of the factor Va chain. By specific to, it is meant that the antibody is capable of readily binding to this region to the exculsion of other regions of factor Va. The amino acid region 307-348 of factor Va heavy chain (42 amino acids, N42R) has been shown to be critical for cofactor activity and may contain a binding site for factor Xa and/or prothrombin ((2001) J. Biol. Chem. 276, 18614-18623). Moreover, it has been shown that that amino acid sequence 323-331 of factor Va heavy chain contains a binding site for factor Xa. (2002) Biochemistry, 41, 12715-12728). An anti-factor V/Va antibody specific to these regions can potentially inhibit cleavage of Factor V into Va, (b) inhibi factor Va binding to phospholipid-bound Xa on platelets; (c) inhibiting the conversion of prothrombin into thrombin, (d) inhibit the release of fibrinopeptide A; (e) inhibi the activation of leukocytes and platelets; and (f) inhibit/reduce the formation of complex phospholipid-Xa-Va, thrombin and fibrin in clinical conditions where the disease pathology is mediated via thrombin production and fibrin formation.

One example of a self-limiting neutralizing monoclonal antibody of this invention is monoclonal antibody NM0035, a murine antibody which can be used for the development of a chimeric or humanized molecule. The NM0035 monoclonal antibody is characterized by a self-limiting inhibitory activity on thrombin formation and FPA formation.

The present invention also includes the use of Fab fragments or F(ab′)₂ fragments derived from monoclonal antibodies directed against the factors V or Va. These fragments are useful as agents having inhibitory activity against thrombin production. A Fab fragment contains the entire light chain and amino terminal portion of the heavy chain. An F(ab′)₂ fragment is the fragment formed by two Fab fragments bound by disulfide bonds. The monoclonal anti-factor V and Va antibodies and other similar high affinity antibodies, provide sources of Fab fragments and F(ab′)₂ fragments which can be obtained by conventional means, e.g., cleavage of the monoclonal antibodies with the appropriate proteolytic enzymes, papain and/or pepsin, or by recombinant methods. These Fab and F(ab′)₂ fragments are useful themselves as therapeutic, prophylactic or diagnostic agents, and as donors of sequences including the variable regions and CDR sequences useful in the formation of recombinant or humanized antibodies as described herein.

The Fab and F(ab′)₂ fragments can be constructed via a combinatorial phage library or via immunoglobulin chain shuffling which are both hereby incorporated by reference in their entirety, wherein the Fd or v_{H} immunoglobulin from a selected antibody is allowed to associate with a repertoire of light chain immuno-globulins, v_{L} (or v_{K}), to form novel Fabs. Conversely, the light chain immunoglobulin from a selected antibody may be allowed to associate with a repertoire of heavy chain immuno-globulins, v_{H} (or Fd), to form novel Fabs Inhibitory factor V or factor Va Fabs can be obtained by allowing the Fd of monoclonal antibodies to associate with a repertoire of light chain immuno-globulins. Hence, one is able to recover neutralizing Fabs with unique sequences (nucleotide and amino acid) from the chain shuffling technique.

The monoclonal anti-factor V and Va antibodies or other antibodies described above may contribute sequences, such as variable heavy and/or light chain peptide sequences, framework sequences, CDR sequences, functional fragments, and analogs thereof, and the nucleic acid sequences encoding them, useful in designing and obtaining various altered antibodies which are characterized by the antigen binding specificity of the donor antibody.

Another desirable protein of this invention may comprise a complete antibody molecule, having full length heavy and light chains or any discrete fragment thereof, such as the Fab or F(ab′)₂ fragments, a heavy chain dimer or any minimal recombinant fragments thereof such as an Fᵥ or a single-chain antibody (SCA) or any other molecule with the same specificity as the selected donor monoclonal antibody. Engineered antibodies may include a humanized antibody containing the framework regions of a selected human immunoglobulin or subtype or a chimeric antibody containing the human heavy and light chain constant regions fused to the coagulation factor antibody functional fragments. A suitable human (or other animal) acceptor antibody may be one selected from a conventional database, e.g., the KABAT. database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain variable framework region for insertion of the donor CDRs. A suitable acceptor antibody capable of donating light chain variable framework regions may be selected in a similar manner. It should be noted that the acceptor antibody heavy and light chains are not required to originate from the same acceptor antibody.

This invention also relates to a method for inhibiting thrombosis in human, which comprises administering an effective dose of an anti-coagulation factor monoclonal antibody having self-limiting neutralizing activity. Preferably, the coagulation factor is from the intrinsic or common coagulation pathway. Most preferably, the anti-coagulation factor monoclonal antibody is an anti-Factor V/Va. The monoclonal antibody can include one or more of the engineered antibodies or altered antibodies described herein or fragments thereof.

Alternatively, acetylsalicylic acid can be administered in combination with the anti-coagulation factor monoclonal antibody. In some cases, combination therapy lowers the therapeutically effective dose of the anti-coagulation factor monoclonal antibody.

The therapeutic response induced by the use of the molecules of this invention is produced by the binding to the respective coagulation factor and the subsequent self-limiting inhibition of the coagulation cascade. Thus, the molecules of the present invention, when in preparations and formulations appropriate for therapeutic use, are highly desirable for persons susceptible to or experiencing abnormal clotting activity associated with, but not limited to, myocardial infarction, unstable angina, atrial fibrillation, stroke, renal damage, pulmonary embolism, deep vein thrombosis and artificial organ and prosthetic implants.

The antibodies, altered antibodies and fragments thereof of this invention may also be used in conjunction with other antibodies, particularly human monoclonal antibodies reactive with other markers (epitopes) responsible for the condition against which the engineered antibody of the invention is directed.

The therapeutic agents of this invention are believed to be desirable for treatment of abnormal clotting conditions from about few hours to about 3 weeks, or as needed. This represents a considerable advances over the currently used anticoagulants heparin and warfarin. The dose and duration of treatment relates to the relative duration of the molecules of the present invention in the human circulation, and can be adjusted by one of skill in the art depending upon the condition being treated and the general health of the patient.

The mode of administration of the therapeutic agent of the invention may be any suitable route which delivers the agent to the host. The antibodies, altered antibodies, engineered antibodies, and fragments thereof, and pharmaceutical compositions of the invention are particularly useful for parenteral administration, i.e., subcutaneously, intramuscularly, intravenously or intra-nasally.

Therapeutic agents of the invention may be prepared as pharmaceutical compositions containing an effective amount of the engineered (e.g., humanized) antibody of the invention as an active ingredient in a pharmaceutically acceptable carrier. Alternatively, the pharmaceutical compositions of the invention could also contain acetysalicylic acid. In the prophylactic agent of the invention, an aqueous suspension or solution containing the engineered antibody, preferably buffered at physiological pH, in a form ready for injection is preferred. The compositions for parenteral administration will commonly comprise a solution of the engineered antibody of the invention or a cocktail thereof dissolved in a pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well-known sterilization techniques (e.g., filtration). The compositions may contain pharmaceutically acceptable auxiliary substances as required to approximate physiological conditions such as pH adjusting and buffering agents, etc. The concentration of the antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of an engineered antibody of the invention. Similarly, a pharmaceutical composition of the invention for intravenous infusion could be made up to contain about 250 ml of sterile Ringer's solution, and about 1 mg to about 30 mg and preferably 5 mg to about 25 mg of an engineered antibody of the invention. Actual methods for preparing parenterally administrable compositions are well known or will be apparent to those skilled in the art and are described in more detail in, for example, “Remington's Pharmaceutical Science”, 15th ed., Mack Publishing Company, Easton, Pa.

It is preferred that the therapeutic agent of the invention, when in a pharmaceutical preparation, be present in unit dose forms. The appropriate therapeutically effective dose can be determined readily by those of skill in the art. To effectively treat a thrombotic or embolic disorder in a human or other animal, one dose of approximately 0.1 mg to approximately 20 mg per kg body weight of a protein or an antibody of this invention should be administered parenterally, preferably i.v. or i.m. Such dose may, if necessary, be repeated at appropriate time intervals selected as appropriate by a physician during the thrombotic response. The compounds of the present invention can be administered in the pure form, as a pharmaceutically acceptable salt derived from inorganic or organic acids and bases, or as a pharmaceutical ‘prodrug.’ The pharmaceutical composition may also contain physiologically tolerable diluents, carriers, adjuvants, and the like. The phrase “pharmaceutically acceptable” means those formulations, which are within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well known in the art, and are described by Berge et al. [14], incorporated herein by reference. Representative salts include, but are not limited to acetate, adipate, alginate, citrate, aspartate, benzoate, benzenesulfonate, chloride, bromide, bisulfate, butyrate, camphorate, camphor sulfonate, gluconate, glycerophosphate, hemisulfate, heptanoate, hexanoate, fumarate, maleate, succinate, oxalate, citrate, hydrochloride, hydrobromide, hydroiodide, lactate, maleate, nicotinate, 2-hydroxyethansulfonate (isothionate), methane sulfonate, 2-naphthalene sulfonate, oxalate, palmitoate, pectinate, persulfate, 3-phenylpropionate, picrate, pivalate, propionate, tartrate, phosphate, glutamate, bicarbonate, p-toluenesulfonate, undecanoate, lithium, sodium, potassium, calcium, magnesium, aluminum, ammonium, tetramethyl ammonium, tetraethylammonium, methylammonium, dimethylammonium, trimethylammonium, triethylammonium, diethylammonium, and ethylammonium, and the like.

The pharmaceutical compositions of this invention can be administered to humans and other mammals enterally or parenterally in a solid, liquid, or vapor form. Enteral route includes, oral, rectal, topical, buccal, and vaginal administration. Parenteral route includes, intravenous, intramuscular, intraperitoneal, intrasternal, and subcutaneous injection or infusion. The compositions can also be delivered through a catheter for local delivery at a target site, via an intracoronary stent (a tubular device composed of a fine wire mesh), or via a biodegradable polymer.

The active compound is mixed under sterile conditions with a pharmaceutically acceptable carrier along with any needed preservatives, exipients, buffers, or propellants. Opthalmic formulations, eye ointments, powders and solutions are also contemplated as being within the scope of this invention. Actual dosage levels of the active ingredients in the pharmaceutical formulation can be varied so as to achieve the desired therapeutic response for a particular patient. The selected dosage level will depend upon the activity of the particular compound, the route of administration, the severity of the condition being treated, and prior medical history of the patient being treated. However, it is within the skill of the art to start doses of the compound at levels lower than required to achieve the desired therapeutic effect and to increase it gradually until optimal therapeutic effect is achieved. The total daily dose of the compounds of this invention administered to a human or lower animal may range from about 0.0001 to about 1000 mg/kg/day. For purposes of oral administration, more preferable doses can be in the range from about 0.001 to about 5 mg/kg/day. If desired, the effective daily dose can be divided into multiple doses for purposes of administration; consequently, single dose compositions may contain such amounts or submultiples thereof to make up the daily dose.

The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated, the severity of the disorder; activity of the specific compound employed; the specific composition employed, age, body weight, general health, sex, diet of the patient; the time of administration, route of administration, and rate of excretion of the specific compound employed, and the duration of the treatment. The compounds of the present invention may also be administered in combination with other drugs if medically necessary.

Compositions suitable for parenteral injection may comprise physiologically acceptable, sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), vegetable oils (such as olive oil), injectable organic esters such as ethyl oleate, and suitable mixtures thereof. These compositions can also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like. Prolonged absorption of the injectable pharmaceutical form can be brought about by the use of agents delaying absorption, for example, aluminum monostearate and gelatin. Proper fluidity can be maintained, for example, by the use of coating materials such as lecithin, by the maintenance of the required particle size in the case of dispersions, and by the use of surfactants. In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly-orthoesters and poly-anhydrides. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Dosage forms for topical administration include powders, sprays, ointments and inhalants. Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol, and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

The present invention also provides pharmaceutical compositions that comprise compounds of the present invention formulated together with one or more non-toxic pharmaceutically acceptable carriers. Compounds of the present invention can also be administered in the form of liposomes. As is known in the art, liposomes are generally derived from phospholipids or other lipid substances. Liposomes are formed by mono- or multi-lamellar hydrated liquid crystals, which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together. Methods to form liposomes are known in the art [15], incorporated herein by reference.

The compounds of the present invention can also be administered to a patient in the form of pharmaceutically acceptable ‘prodrugs.’ The term “pharmaceutically acceptable prodrugs” as used herein represents those prodrugs of the compounds of the present invention which are, within the scope of sound medical judgment, suitable for use in contact with the tissues of humans and lower animals without undue toxicity, irritation, allergic response, and the like, commensurate with a reasonable benefit/risk ratio, and effective for their intended use, as well as the zwitterionic forms, where possible, of the compounds of the invention. Prodrugs of the present invention may be rapidly transformed in vivo to the parent compound of the above formula, for example, by hydrolysis in blood. A thorough discussion is provided by Higuchi and Stella, (year or ref number?) incorporated herein by reference.

The Examples which follow are presented to describe preferred embodiments and utilities of the invention and are not meant to limit the invention unless otherwise stated in the claims appended hereto. The description is intended as a non-limiting illustration, since many variations will become apparent to those skilled in the art in view thereof. It is intended that all such variation within the scope and spirit of the appended claims be embraced thereby. Changes can be made in the composition, operation, and arrangement of the method of the present invention described herein without departing from the concept and scope of the invention as defined in the claims.

### EXAMPLE 1

### Anti-Factor V/Va Monoclonal Antibodies Bind Factor Va

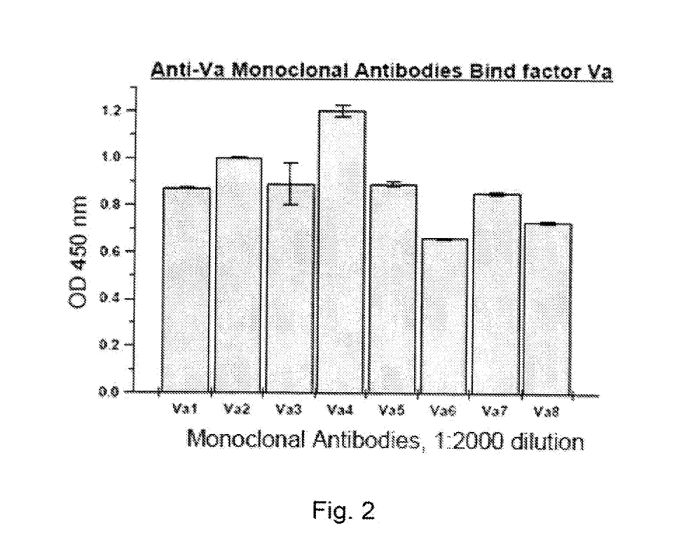

Eight commercially obtained monoclonal antibodies to factor V or Va were tested for binding to Substrate-bound factor Va. The binding data revealed that the factor V/Va monoclonal antibodies bind factor Va with equimolar affinity. In a typical assay, ELISA plates were coated with 20 ng of Factor Va in 50 ul PBS per well and incubated overnight in cold. The plates were treated with 1% BSA in PBS for lhour to block the non-specific sites on ELISA plate. Anti-Factor V/Va monoclonal antibodies: Anti-Bovine Factor V (HTI, LOT#L0811 CAT#ABV-5104, Heavy Chain), Anti-Bovine Factor V (HTI, LOT#K0109 CAT#ABV-5105, Light Chain), Anti-Bovine Factor V (HTI, LOT#J0731 CAT#ABV-5106, Heavy Chain), Anti-Bovine Factor V (HTI, LOT#L0918 CAT#ABV-5107, Light Chain), Anti-Human Factor V (HTI, LOT#L0213 CAT#AHV-5101, Light Chain), Anti-Human Factor V (HTI, CAT#AHV-5108, Light Chain), Anti-Human Factor V (HTI, LOT#L0806 CAT#AHV-5112, Light Chain), Anti-Human Factor V (HTI, LOT#N1011 CAT#AHV-5146, Heavy Chain) in blocking solution at 1:2000 dilutions were incubated with substrate bound factor Va. Following a one hour incubation at room temperature, the plate was rinsed and the bound anti-Factor V/Va monoclonal antibodies were detected with peroxidase-conjugated goat anti-mouse antibody (Sigma Chemical Company) at 1:2000 dilution. The plate was washed and incubated with 100 ul of TMB sustrate for 10 minutes. The plate was read at 450 nm after quenching with 100 ul aliquots of 1 M phosphoric acid. As shown in FIG. 2, all monoclonal antibodies bind factor V.

### EXAMPLE 2

### Anti-Factor Va Inhibits Thrombin Generation in Tissue Factor Pathway Assay

The binding data above revealed that factor V monoclonal antibodies bind factor Va. All eight anti-factor V monoclonal antibodies that bound factor Va were also tested for their ability to prevent thrombin production in citrated human plasma. Since factor Va is the critical component of the prothrombinase enzyme (a central component of the coagulation cascade), it was of interest to us to examine the ability of anti-factor V monoclonal antibodies in preventing thrombin generation in extrinsic pathway (tissue factor pathway). The final end product of the tissue factor pathway is the generation of thrombin. Previous studies have demonstrated that Tissue Factor/CaCl₂ (Innovin, Dade Behring, PT reagent) serves as a potent enzyme for extrinsic pathway activation. Citrate (5% human plasma in TB S buffer) human plasma (100 μl) was mixed with 75 μl of 0.5 mg/ml concentration of S-2238 (Chromogenix). Innovin (50 μl) was added to the mixture and the kinetic reaction was allowed to proceed at 37° C. The progressive increase in the colorimetric signal at 405 was followed over time. The effect of the anti-factor V/Va blocking monoclonal antibody on the thrombin formation was evaluated by adding a fixed concentration of the blocking antibody to a fixed concentration of plasma (5% citrated plasma in TBS buffer containing 1% bovine serum albumin (BSA)). The inhibitory effect of the monoclonal anti-factor V/Va on thrombin formation was determined using the chromogenix assay as described.

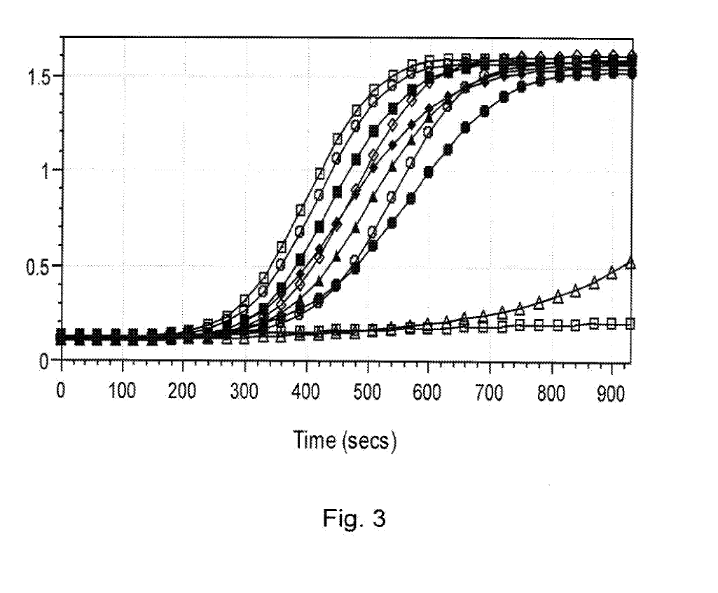

As demonstrated in FIG. 3, thrombin formation was completely inhibited by only one of eight anti-factor V monoclonal antibody recognized as Anti-Human Factor V (HTI, LOT#L0213 CAT#AHV-5101, Light Chain). While the antibody known to inhibit the coagulation (Anti-Bovine Factor V (HTI, LOT#L0811 CAT#ABV-5104, Heavy Chain) showed no effect of thrombin inhibition. This experiment demonstrates that the assay is able to select the most potent anti-factor V monoclonal antibody that prevents thrombin formation. In FIG. 3 are shown seven anti-factor V monoclonal antibodies that do not inhibit thrombin production. The first two lines are duplicate controls. The seven lines represent monoclonal antibodies. The bottom two lines are; Anti-Human Factor V (HTI, LOT#L0213 CAT#AHV-5101, Light Chain and heparin control (the bottommost line).

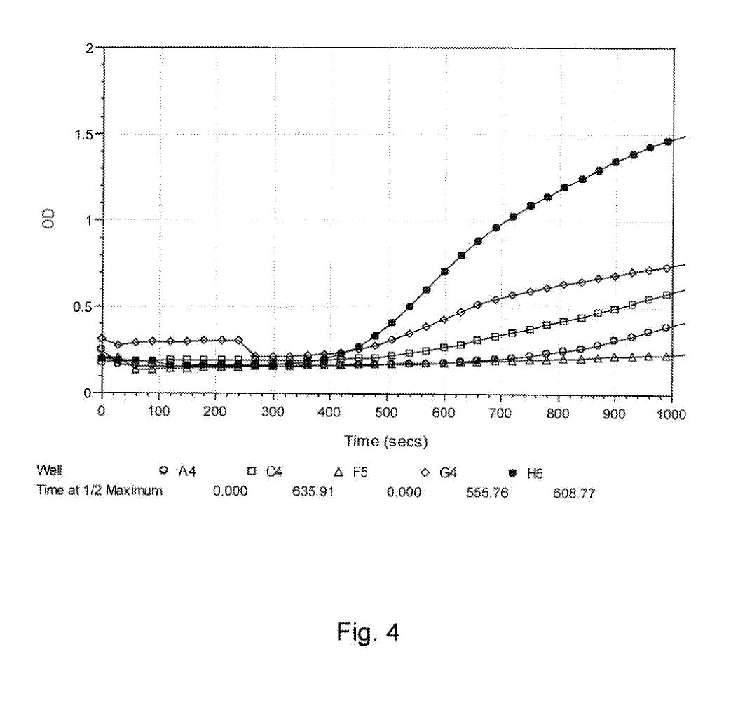

The selected monoclonal antibody, Anti-Human Factor V (HTI, LOT#L0213 CAT#AHV-5101, Light Chain) was again evaluated at different doses. As shown in FIG. 4, the monoclonal antibody inhibition of tissue factor-mediated activation of thrombin formation is dose dependent with 100 ug of the monoclonal achieving greater than 95% inhibition. The first line from the top represents the untreated control.

### EXAMPLE 3

### Anti-Factor Va Inhibits Thrombin Generation in Intrinsic Pathway (FSL) Assay

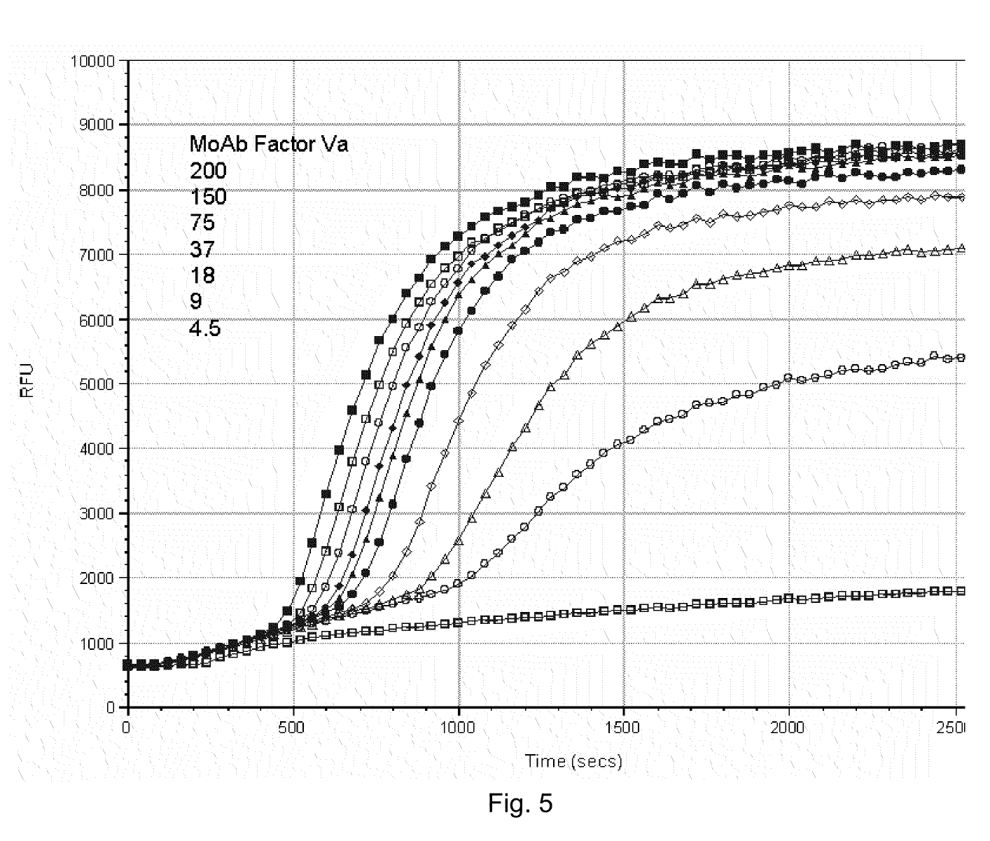

The thrombin data shown in example 2 revealed that an anti-factor factor V monoclonal antibody to factor V binds factor Va and prevents the extrinsic pathway of coagulation as shown by inhibition of thrombin production. Since both extrinsic and intrinsic coagulation pathways converge at the prothrombinase level, it was of interest to us to examine the ability of the selected anti-factor V monoclonal antibody to inhibit the intrinsic coagulation pathway. The final end product of the intrinsic pathway is the generation of thrombin. In a typical assay, 80 ul of citrated plasma (final concentration 20%) in Tris buffered saline containing 1 mMCaCl₂ was mixed with 50 ul of Fluorescent Thrombin substrate “Technothrombin”. A 50 ul aliquot of FSL (aPTT reagent from Dade Behring) was added to the mixture. The plate was incubated at 37° C. and progressive increase in fluorescence was recorded with time. To evaluate the effect of the selected monoclonal antibody (from Example 2) in this assay, the antibody was tested at 200 ug/ml, 150 ug/ml, 75 ug/ml, 37.5 ug/ml, 18.75 ug/ml, 9.38 ug/ml, 4.5 ug/ml, and 2.25 ug/ml. The data was compared to heparin and untreated controls. As shown in FIG. 5, the antibody demonstrated a dose dependent inhibition of thrombin production in this assay. These data suggest that the monoclonal antibody prevents factor V function in the formation of prothrombinase. The figure shows that 200 ug/ml of the monoclonal only provided 50% inhibition of thrombin production.

### EXAMPLE 4

### Factor Va Binds Substrate-Bound Xa with High Affinity

Polystyrene microtiter plates were coated with human factor Xa in TBS (Tris Buffered Saline): overnight at 4° C. After aspirating the factor Xa solution, wells were blocked with TBS containing 0.5% HSA (Human Serum Albumin, Sigma Chemical Company, St. Louis, Mo., Cat. No. A9511) for 2 hours at room temperature. Wells without factor Xa coating served as background controls. Aliquots of human factor V/Va at varying concentrations in blocking solution (containing 0.1 mM calcium) were added to the wells. Following a 2 h incubation at room temperature, the wells were extensively rinsed with PBS.

Xa-bound Va was detected by the addition of peroxidase-conjugated mouse monoclonal anti-human factor Va antibody (detection antibody) at 1:2000 dilution in blocking solution, which was allowed to incubate for 1 h at room temperature. After washing the plates with TBS, 100 ul aliquots of TMB substrate (Kirkegaard & Perry Laboratories, Gaithersburg, Md.) were added. After incubation for 10 min at 25° C., the reaction of TMB was quenched by the addition of 100 μl of phosphoric acid, and the plate was read at 450 nm in a microplate reader (e.g., SPECTRA MAX 250, Molecular Devices, Sunnyvale, Calif.). The estimated K_{d} of factor Va binding to Xa was based on the concentration of factor Va at 50% maximal binding (Microcal Origin Program).

As shown in FIG. 6, human factor Va binds to Xa, which has been immobilized onto microtiter plate wells. The apparent binding constant from these data, defined as the concentration of factor Va needed to reach half-maximal binding, is approximately 2 nM. We have also evaluated the ability of anti-factor Va monoclonal antibodies to inhibit the binding of factor Va to factor Xa. Anti-factor Va monoclonal antibody was added to a fixed concentration of factor Va in blocking solution. This reaction mixture was incubated with Xa to evaluate inhibition of Va binding to Xa. As shown in FIG. 6 (bottom panel), factor Va binding to factor Xa was inhibited by the selected factor Va monoclonal antibody.

### EXAMPLE 5

### Binding of Plasma Factor Va to Phospholipid Bound Xa

Polystyrene microtiter plates were coated with factor Xa (Haemtech, Vermont) in Tris buffered saline overnight at 4° C. After aspirating the factor Xa solution, wells were blocked with Tris Buffered Saline (TBS) containing 1% human serum albumin (HSA) (Sigma Chemical Company, St. Louis, Mo.) for 2 h at room temperature. Wells without Xa coating served as background controls. Aliquots of plasma containing factor Va were added and plates were allowed to sit for 2 h in to allow factor Va binding to substrate-bound Xa. Factor Va bound to Xa was detected by the addition of peroxidase-conjugated mouse monoclonal anti-human factor Va antibody (detection antibody) at 1:2000 dilution in blocking solution, which was allowed to incubate for 1 h at room temperature. The plate was again rinsed thoroughly with TBS, and 100 μl of 3,3′,5,5′-tetramethyl benzidine (TMB) substrate (Kirkegaard & Perry Laboratories, Gaithersburg, Md., Cat. No. A50-65-00) was added. After incubation for 10 min at 25° C., the reaction of TMB was quenched by the addition of 100 μl of phosphoric acid, and the plate was read at 450 nm in a microplate reader (e.g., SPECTRA MAX 250, Molecular Devices, Sunnyvale, Calif.). The estimated Kd of Va binding to Xa was based on the concentration of Va at 50% maximal binding (Microcal Origin Program).

As shown in FIG. 6, human factor Va binds to Xa in presence of physiological milieu, which has been immobilized onto microtiter plate wells. The apparent binding constant from these data, defined as the concentration of factor Va needed to reach half-maximal binding, is approximately 2 nM.

### REFERENCES

1. Heemskerk J W, Bevers E M, Lindhout T. 2002. Platelet activation and blood coagulation. Thromb Haemost 88: 186-93
2. Hemker H C, van Rijn J L, Rosing J, van Dieijen G, Bevers E M, Zwaal R F. 1983. Platelet membrane involvement in blood coagulation. Blood Cells 9: 303-17
3. Ahmad S S, London F S, Walsh P N. 2003. The assembly of the factor X-activating complex on activated human platelets. J Thromb Haemost 1: 48-59
4. Krishnaswamy S, Russell G D, Mann K G. 1989. The reassociation of factor Va from its isolated subunits. J Biol Chem 264: 3160-8
5. Bradford H N, Annamalai A, Doshi K, Colman R W. 1988. Factor V is activated and cleaved by platelet calpain: comparison with thrombin proteolysis. Blood 71: 388-94
6. Nesheim M E, Mann K G. 1979. Thrombin-catalyzed activation of single chain bovine factor V. J Biol Chem 254: 1326-34
7. Nesheim M E, Kettner C, Shaw E, Mann K G. 1981. Cofactor dependence of factor Xa incorporation into the prothrombinase complex. J Biol Chem 256: 6537-40
8. Nesheim M E, Taswell J B, Mann K G. 1979. The contribution of bovine Factor V and Factor Va to the activity of prothrombinase. J Biol Chem 254: 10952-62
9. Rosing J, Tans G, Govers-Riemslag J W, Zwaal R F, Hemker H C. 1980. The role of phospholipids and factor Va in the prothrombinase complex. J Biol Chem 255: 274-83
10. Walenga J M, Jeske W P, Hoppensteadt D, Fareed J. 2003. Factor Xa inhibitors: today and beyond. Curr Opin Investig Drugs 4: 272-81
11. Katira R, Chauhan A, More R S. 2005. Direct thrombin inhibitors: novel antithrombotics on the horizon in the thromboprophylactic management of atrial fibrillation. Postgrad Med J 81: 370-5
12. L. Badimon BJMaJJB, Thrombin in arterial thrombosis, Haemostasis 24 (1994), pp. 69-80.
13. J. Nappi Tbotiacs, Pharmacotherapy 22 (2002), pp. 90S-96S.
14. Gulseth M P. 2005. Ximelagatran: an orally active direct thrombin inhibitor. Am J Health Syst Pharm 62: 1451-67
15. Foster W B, Tucker M M, Katzmann J A, Mann K G. 1983. Monoclonal antibodies selective for activated Factor V Immunochemical probes for structural transitions occurring during the thrombin-catalyzed activation of the procofactor. J Biol Chem 258: 5608-13
16. Foster W B, Katzmann J A, Miller R S, Nesheim M E, Mann K G. 1982. Monoclonal antibodies selective for the functional states of bovine factor V and factor Va. Thromb Res 28: 649-61

Having described the invention we claim:

1. A process of inhibiting the adverse effects of coagulation pathway activation products in a mammal comprising administering to the mammal an amount of anti-factor V/Va antibody that is effective to inhibit formation of common coagulation pathway activation product.

2. The process of claim 1 wherein the amount of the anti-Factor V/Va antibody is effective to inhibit formation of Thrombin.

3. The process of claim 1 wherein the amount of the anti-Factor Va antibody is effective to inhibit formation of fibrin.

4. The process of claim 1 wherein the amount of the anti-Factor V/Va antibodies inhibit the conversion of Factor V into Va

5. The process of claim 1 wherein the anti-factor Va antibody is specific to the heavy chain of factor Va.

6. The process of claim 1 wherein the anti-factor V/Va agent is an anti-factor V/Va antibody specific to the light chain of factor V/Va.

7. The process of claim 1 wherein the anti-factor V/Va antibody is specific to the peptide region 307 through 348.

8. The process of claim 1 wherein the anti-factor Va antibody lacks the ability to activate Fc gamma receptors.

9. The process of claim 1, wherein the antibody is a a chimeric, recombinant, de-immunized, humanized, or human antibody.

10. The process of claim 1 wherein said antibody inhibits cleavage of prothrombin into thrombin

11. The method of claims 1 wherein said V/Va antibody comprises F_{ab}, F_{(ab)2},Fᵥ,scFv

12. The method of claim 1, wherein the antibody inhibits clot formation in a blood vessel

13. The process of claim 1, wherein the anti-factor V/Va antibody inhibits the extrinsic pathway of coagulation.

14. The process of claim 1 wherein the anti-factor V/Va antibody inhibits the intrinsic pathway of coagulation

15. A method of claim 1, wherein the said anti-factor V/Va antibody can be selected from the group consisting of myocardial infarction, ischemia/reperfusion, stroke, acute respiratory distress syndrome (ARDS) injury, cardiopulmonary bypass inflammation, extracoporeal circulation, percutaneous transluminal coronary angioplasty (PTCA), artificial organs, shunts, prostheses, arterial fibrillation, unstable angina, pulmonary embolism, Deep Vein Thrombosis (DVT), transplant rejection, multiple sclerosis, myasthenia gravis, pancreatitis, rheumatoid arthritis, Alzheimer's disease, asthma, thermal injury, anaphylactic shock, bowel inflammation, urticaria, angioedema, vasculitis, Sjogren's syndrome, lupus erythromatosus, and membranous nephritis, vascular stenosis and restenosis.

16. The method of claim 15, wherein said disease is myocardial infarction.

17. The method of claim 15, wherein said disease is ischemia/reperfusion injury.

18. The method of claim 15, wherein said disease is a stroke

19. The method of claim 15, wherein said disease is cardiopulmonary bypass inflammation

20. The method of claim 15, wherein said disease is percutaneous transluminal coronary angioplasty (PTCA)

21. The method of claim 15, wherein said disease is unstable angina

22. The method of claim 15, wherein said disease is deep vein thrombosis.

23. The method of claim 15, wherein said disease is pulmonary embolism.

24. The method of claim 15, wherein said disease is artificial organs.

25. A method of treating diseases resulting from coagulation activation comprising the steps of:
(a) Selecting an inhibitor molecule anti-factor V/Va antibody molecule with antigenic determinant on light and heavy chain of factor V/Va.
(b) Establishing by ex vivo assay procedures that said inhibitor inhibits thrombin production
(c) Establishing by in vitro assay procedures that said inhibitor further prevents factor Va binding to factor Xa or phospholipid-bound factor Xa, prevents formation of thrombin, and prevents formation of fibrin, reduces activation of platelets, and leukocytes.
(d) Delivering an effective amount of said inhibitor to an individual through subcutaneous, intravenous, intranasal, intratracheal, intraspinal, intracranial, or oral administration.
